(19) United States
(12) Patent Application Publication
(10) Pub. No.: US 2007/0293759 A1
(43) Pub. Date: Dec. 20, 2007

(54) Doppler Flow Measurement Apparatus

(75) Inventors: George Eilers, Evergreen, CO (US); Wes Weber, Golden, CO (US); Edward Spence, Bronx, NY (US)

Correspondence Address: DARBY & DARBY P.C., P.O. BOX 770, Church Street Station, New York, NY, 10008-0770, US

(73) Assignee: NEPHROS, INC., New York, NY (US)

(21) Appl. No.: 11/574,605

(22) PCT Filed: Sep. 2, 2005
(86) PCT No.: PCT/US05/31363

§ 371 Date: Mar. 2, 2007

Related U.S. Application Data
(60) Provisional application No. 60/607,112, filed on Sep. 3, 2004.

(51) Int. Cl. A61B 8/06

(52) U.S. Cl. 600/454

(57) ABSTRACT

The present invention relates to a measurement of a flow by the use of Doppler ultrasound, and in particular, by analyzing the spectrum of the reflected ultrasonic signal. The reflected signal is mixed electronically with the transmitted signal in a product detector, demodulator or similar component (250) to produce a signal that is shifted in frequency. In another embodiment, the method includes analyzing the spectrum (280, 300) of the reflected ultrasonic beams by performing the step of: (a) determining a maximum frequency of the detector output signal which is representative of a maximum particle velocity; and (b) calculating a total flow of the particles based on the maximum particle velocity.

### TECHNICAL FIELD

The present invention relates to measuring devices and more particularly, relates to an accurate, non-invasive apparatus that can measure the flow of a mixture of liquid and particles in a conduit, such as an extracorporeal blood circuit.

### BACKGROUND

A number of practical situations require an accurate, non-invasive measurement of flow of a mixture of liquid and particles. An example of this is a circuit containing whole blood which comprises a mixture of serum liquid and formed elements. Such a flow measurement in the extracorporeal blood circuit would be of value during a hemodialysis or hemodiafiltration procedure. Typically, the flow is confined to tubing made of PVC or other flexible polymer material and interrupting the tubing to insert a flow measuring device is impractical.

There are a number of different techniques that can be used to establish the speed of flow of a fluid within a conduit. For example, one technique for establishing the speed of flow of blood in blood vessels is based on measuring the ultrasonic Doppler effect of the fluid flowing in the conduit. Such a method is disclosed in U.S. Pat. No. 3,675,192, which is hereby incorporated by reference in its entirety. The system disclosed in the '192 patent includes an ultrasonic transmitter and as receiver for receiving the ultrasonic energy (of the echo signals) reflected off the flowing blood, as well as means for determining a value proportional to the speed of flow of blood from the transmission and reception frequencies. Similar devices that operate on detecting the Doppler effect are set forth in U.S. Pat. Nos. 3,741,014; 4,391,149 and 4,809,703, which are hereby incorporated by reference in their entirety.

The above identified prior art Doppler flow measuring devices analyze the spectrum of the reflected signals in an attempt to determine the total flow or the particle velocity or both. These devices product only qualitative and inaccurate results for reasons described in more detail below.

More specifically, Fahrbach (U.S. Pat. No. 3,675,192) discloses a Doppler flow measurement device that analyzes the output of the demodulator by passing it through an array of electrical filters, each tuned to a particular part of the frequency spectrum. The resulting outputs of the filter array are then fed to a peak detector to yield a set of voltages that are proportional to the spectral amplitudes at the filters' respective center frequencies. Fahrbach proceeds to combine the set of voltages in two different ways. In one way, the voltages are multiplied by a frequency-proportionate weighing factor and then summed. In the other, the voltages are summed with a unity weighting factor. The quotient of the frequency-weighted sum by the unity-weighted sum is then expected to be representative of the total flow of particles in the liquid.

The theory upon which Fahrbach's invention is based postulates that the spectral amplitude of the reflected signal at a given frequency is proportional to the number of particles flowing at the corresponding particle velocity. Consequently, summing the spectral amplitudes weighted by their frequencies should yield a result that is representative of the total particle flow rate.

The approach used by Fahrbach fails because the spectral amplitude of the net reflection produced by a large number of particles is not proportional to the number of particles because it is the result of the random combination of phases and amplitudes of the particles.

Accordingly, there is a need for an alternative method for determining the speed of flow (flow rate) of a fluid in a way which overcomes the above deficiencies that are associated with the prior art.

### SUMMARY

The present invention relates to a measurement of a flow, as described below, by the use of Doppler ultrasound. The present invention measures flow by analyzing the spectrum of the reflected ultrasonic signal. Using conventional techniques, the reflected signal is mixed electronically with the transmitted signal in a product detector, demodulator, or similar component to product a signal that is shifted in frequency.

In one embodiment, the method of accurately and non-invasively measuring a flow of mixture of liquid and particles includes the step of analyzing the spectrum of the reflected ultrasonic beams by performing the steps of (a) calculating a spectral intensity by performing a discrete Fourier transform (FT) of an output of the detector by processing the results of the Fourier transform to obtain the spectral intensity which is the square of a spectral amplitude of the FT results and then (b) multiplying the spectral intensity by the corresponding frequency of the spectral amplitude of the output and then (c) summing all frequencies of the results of the Fourier transform to yield a value that is proportional to a total flow of the particles.

In another embodiment, the method of accurately and non-invasively measuring a flow of mixture of liquid and particles includes the step of analyzing the spectrum of the reflected ultrasonic beams by performing the step of: (a) determining a maximum frequency of the detector output signal which is representative of a maximum particle velocity; and (b) calculating a total flow of the particles based on the maximum particle frequency. The maximum frequency of the detector output signal can be determined by performing a discrete Fourier transform of the detector output signal to yield an FT output; and calculating a spectral edge by detecting the frequency where a spectral amplitude of the FT output substantially decreases over a short period of time, with the spectral edge representing the maximum particle velocity.

In yet another embodiment of the present invention, the spectral intensity is calculated by performing a discrete Fourier transform of the product detector's output. The discrete Fourier transform results are then processed to obtain the spectral intensity. The spectral intensity is multiplied by a frequency-proportionate weighting factor and summed to produce a numerator of a quotient. The spectral intensity is also summed with a unity weighting factor to produce the denominator of the quotient. The quotient is then taken as representative of the total flow of particles.

Other features and advantages of the present invention will be apparent from the following detailed description when read in conjunction with the accompanying drawings.

### BRIEF DESCRIPTION OF THE DRAWING FIGURES

The foregoing and other features of the present invention will be more readily apparent from the following detailed description and drawings figures of illustrative embodiments of the invention in which:

FIG. 1 is a diagrammatic schematic of a conventional Doppler flow measurement apparatus;

FIG. 2 is a diagrammatic schematic illustrating the processing steps of a conventional Doppler flow measurement apparatus for producing a Doppler output signal;

FIG. 3 is a diagrammatic schematic illustrating the processing steps of a Doppler flow measurement apparatus for calculating speed of flow of a fluid (flow rate) through a conduit according to a first embodiment of the present invention;

FIG. 4 is a diagrammatic schematic illustrating the processing steps of a Doppler flow measurement apparatus for calculating speed of flow of a fluid (flow rate) through a conduit according to a second embodiment of the present invention; and

FIG. 5 is a graph showing the output of a fast Fourier transform (FFT) program which operates to calculate the spectral amplitude distribution of a Doppler output and provides a graph of signal amplitude vs. signal frequency.

### DETAILED DESCRIPTION OF PREFERRED EMBODIMENTS

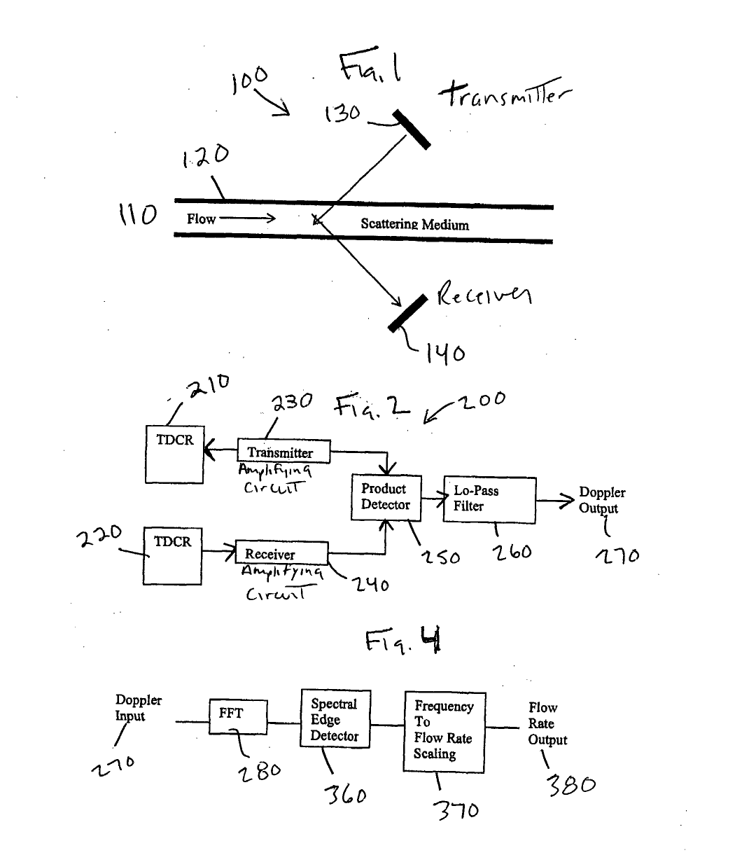

FIG. 1 illustrates a conventional flow measurement apparatus 100 that provides an accurate, non-invasive measurement of a flow 110 of a mixture of liquid and particles within a conduit (tubing) 120 by use of Doppler ultrasound. It is known that particles in a liquid reflect ultrasound and that moving particles provide a reflection, whose frequency is shifted by an amount proportional to their velocity. A beam of ultrasound, typically with a frequency of between about 2 MHz and about 5 MHz, is directed at an angle to the flow, indicated at 110, by placing an angled transducer (transmitter) 130 adjacent to the conduit 120.

The reflected ultrasound can be sensed by another transducer (receiver) 140 that is located adjacent the tubing 120 and spaced from the transducer 130. For example, the first transducer 130 can be orientated at a predetermined degree angle from the second transducer 140. However, this angle is merely illustrative and not limiting in any way. For example, the angle between the two components 130, 140 can be equal to or greater than or less than 90 degrees, with FIG. 1 showing an angle of about 90 degrees. Alternatively, the ultrasound beam can be pulsed and the same angled transducer can be used to both transmit the beam and to receive the reflected signal.

The second transducer (receiver) 140 receives the ultrasonic energy scattered by the flow medium 110 (e.g., particles in blood), with the received ultrasound having a Doppler frequency shift that is proportional to the flow velocity of the scattering medium. The particle velocity in the liquid stream is known to vary with the location in the stream. In one condition, where the flow is laminar and Newtonian and the conduit 120 carrying the stream 110 has a circular cross-section, the particle velocity is known to have a parabolic profile. The particle velocity is greatest at the center of the conduit, falling parabolically to zero at the walls of the conduit 120. In this condition, the total flow through the conduit 120 can be calculated from the maximum particle velocity, and the relationship between the total flow and maximum particle velocity is linear.

In another condition, the flow is laminar and non-Newtonian and the conduit 120 carrying the stream 110 has a non-circular cross-section. This condition is typical of blood flowing in a flexible tubing that is tightly clamped in a rectangular fixture. In this condition, the particle velocity varies with location, but not parabolically. Also, the particle velocity profile varies with the total flow rate. The total flow can also be calculated from the maximum particle velocity, but the relationship, while predictable, is not linear.

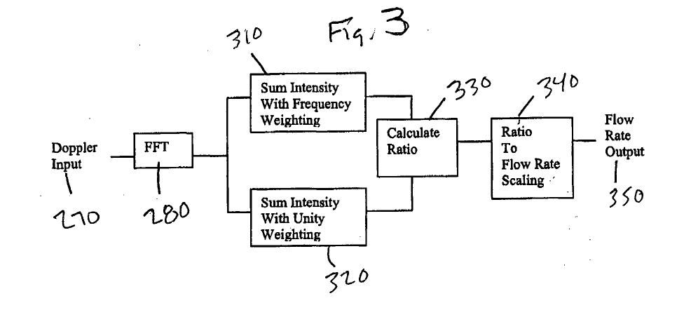

FIG. 2 is a schematic diagram that illustrates the processing steps of a conventional Doppler flow measurement apparatus for producing a Doppler output signal. More specifically, FIG. 2 shows a system 200 for establishing the flow rate (speed of flow) of the stream 110 within the conduit 120 using an ultrasonic Doppler frequency shift method. The system 200 includes a first transducer 210 that is associated with the transmitter 130 (labeled in FIG. 2 as “TDCR”) and a second transducer 220 that is associated with the receiver 140 (also labeled TDCR).

The system 200 also includes a transmitter amplifying circuit 230 and a receiver amplifying circuit 240. The transmitter amplifying circuit 230 generates a high-amplitude signal that is sent to the first transducer 210 causing the first transducer 210 to emit an ultrasound beam (energy) as described with reference to FIG. 1. An arrow shows the delivery of the signal from the circuit 230 to the first transducer 210. The receiver amplifying circuit 240 receives the reflected signal from the second transducer 220 and amplifies it to a level that is sufficient and appropriate for a product detector 250. The product detector 250 combines the signal from the transmitter 120 that represents the signal that is fed to the first transducer 210 with the amplified signal from the receiver 130 and operates to produce as an output a signal whose amplitude is proportional to the signal received from the receiver amplifying circuit 240, but whose frequency is the difference between transmitter frequency and the frequency of the signal received by the second transducer 220. Arrows are shown in FIG. 2 to indicate the delivery of the signals to the product detector 250.

A low pass filter 260 is provided and functions as a standard low pass filter in that it filters and eliminates all frequency components that are greater than the highest expected Doppler frequency shift. The low pass filter 260 is needed since the product detector 250 also emits signals that have frequencies higher than the highest expected Doppler frequency. For example, the transmitter 120 can emit a signal that has a frequency of 4 MHz and the reflected signal received by the receiver 130 has a frequency of 4 MHz but is shifted by between about 0 and 3000 Hz due to the Doppler effect. The product detector 250 receives both of these signals and then outputs a number of different signals of varying frequencies. One of the signals that is output by the product detector 250 is the signal that corresponds to and represents the Doppler shift, namely, a signal having a frequency between about 0 and 3000 Hz. The low pass filter 260 thus operates to filter out any signals that have frequencies greater than the Doppler shift frequency of between about 0 and 3000 Hz. The Doppler output signal, generally indicated at 270, generated after low pass filtering is performed thus has a frequency of between about 0 and 3000 Hz depending upon the magnitude of the Doppler shift which in turn depends on the velocity of the scattering medium (flow 110).

The system and method of the present invention measure flow by analyzing the spectrum of the reflected ultrasonic signal. Following the method described with reference to FIG. 2 and as described in the above-referenced patents, the reflected signal is mixed electronically with the transmitted signal in a product detector, demodulator, or similar component 250 to produce a signal (Doppler output signal 270) that is shifted in frequency. In this arrangement, reflections from stationary objects produce a zero-frequency or dc output. Reflections from a moving object, such as the particles in a flowing stream (flow 110) produce a frequency proportional to their velocity. If a product detector is used, the amplitude of the frequency-shifted signal from an individual moving particle is proportional to the strength of its ultrasound reflection.

As previously mentioned, the approach used by Fahrbach fails because the spectral amplitude of the net reflection produced by a large number of particles is not proportional to the number of particles because it is the result of the random combination of phases and amplitudes of the particles. In such a random combination, the spectral amplitude of the net reflection is instead proportional to the square root of the number of particles. The spectral intensity, on the other hand, which is the square of the spectral amplitude, is proportional to the number of particles. The present applicants have thus realized this critical deficiency and have proposed a solution and accordingly, the system and method of the present invention use and perform operations on the spectral intensity as opposed to the spectral amplitude as described by Fahrbach.

In one embodiment of the present invention which is illustrated in FIG. 3, the spectral intensity is calculated by performing a discrete Fourier transform of the product detector's output (signal 270). As set forth in the diagrammatic flow chart of FIG. 3, the Doppler input (which is represented by signal 270 of FIG. 2) is delivered to a processor or software 280 which is capable of performing a fast Fourier transform (FFT) operation and producing a spectral intensity output. As is known, a fast Fourier transform is an efficient algorithm to compute the discrete Fourier transform (DFT) and its inverse. FFTs are of great important to a wide variety of application, from digital signal processing, as in the present invention. The results of the FFT 280 can be graphically depicted on a graph 300, such as the one illustrated in FIG. 5, which plots the signal amplitude verse the signal frequency of the Doppler input signal. In this manner, a range of the Doppler spectrum of the input signal (signal 270) is calculated and graphically depicted in graph 300.

The spectral distribution depicted in graph 300 also corresponds to the distribution of the velocities of the particles that are present in the stream 110. It will be understood that the graph 300 thus is representative of the contributions of the different particles of the scattering medium (stream 110) which corresponds to the different velocities of the particles in the scattering medium. For example, in the case of blood, the predominant particles that scatter (reflects) the signal are red blood cells, with the particles having a higher velocity having a higher associated frequency.

The discrete Fourier transform results are then processed to obtain the spectral intensity which is the basis for calculating the flow rate output according to the present invention. Different from the spectral amplitude, the spectral intensity represents the square of the spectral amplitude. The spectral intensity is multiplied by a frequency-proportionate weighting factor and summed to product a numerator of a quotient as indicated at step 310 in FIG. 3. The spectral intensity is also summed with a unity weighting factor to product the denominator of the quotient as indicated at step 320 in FIG. 3. The quotient is then taken as representative of the total flow of particles as indicated at step 330 in FIG. 3.

In other words, the amplitude of the output of the FFT is taken at each frequency and squared to produce the spectral intensity at that frequency which in turn is multiplied by the frequency. Then the resulting products for all frequencies of the output of the FFT are summed and this sum is used as the numerator of a quotient. The same process is also performed without multiplying by the frequency and that sum is used as the denominator of the quotient. The value of the quotient is proportional to the volume flow rate of the stream 110 in the conduit 120. It will be appreciated that this is different than using the spectral amplitude as a basis for the calculation as in Fahrbach in place of the spectral intensity as described above.

The ratio-to-flow rate scaling that is indicated in step 340 of FIG. 3 is merely a typical scaling operation where the frequency-to-flow-rate conversion is calibrated. For example, if a ratio of 1.5 corresponded to a flow rate of 100 ml/minute, then a ratio of 3.0 would correspond to a flow rate of 200 ml/minute.

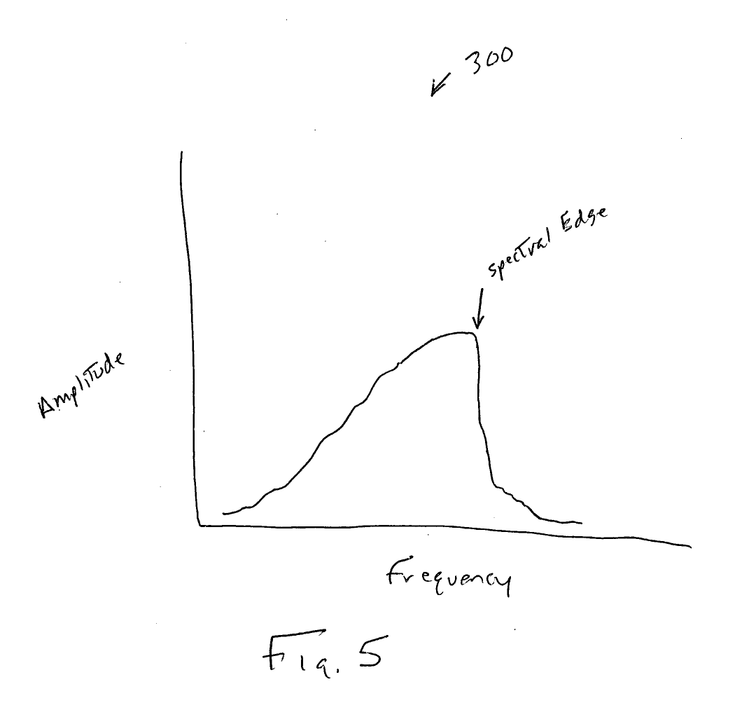

In another embodiment of the present invention that is illustrated in FIG. 4, the spectral amplitude or intensity is analyzed to determine the maximum frequency of the product detector's output signal. Each frequency component contained in the Doppler signal's spectrum corresponds to a particle velocity in the flowing liquid, and the particle velocity has a definite maximum at some point in the flowing stream for each flow rate. This maximum frequency component contained in the Doppler signal's spectrum therefore corresponds to the maximum particle velocity. As described above, the total particle flow rate is reliably, although not linearly for a non-Newtonian fluid, related to the maximum particle velocity and therefore to the maximum frequency of the product detector's output signal.

FIG. 4 illustrates this second embodiment and is described in terms of analyzing the spectral amplitude; however, as mentioned above, the same analysis can be performed on the spectral intensity rather than the spectral amplitude. The graph 300 (FIG. 5) illustrates a typical spectral amplitude frequency distribution. Both spectral intensity and spectral amplitude have similar but differently shaped spectral frequency distributions from which the frequency corresponding to the maximum particle velocity can be obtained by noting the location of the spectral edge, the frequency where the intensity or the amplitude drops off sharply. The spectral edge detector 360 locates the position of the spectral edge in the FFT output.

Once the spectral edge is detected by the detector 360, the detector 360 outputs a signal, which corresponds to the spectral amplitude frequency where the spectral edge is located, which then undergoes a frequency-to-flow-rate scaling operation as indicated at step 370 (scaling operation where the frequency to flow rate is calibrated). The result of performing the method/operations of FIG. 4 is a flow rate output 380 which is a volume of flow per unit of time.

While exemplary drawings and specific embodiments of the present invention have been described and illustrated, it is to be understood that the scope of the present invention is not to be limited to the particular embodiments discussed. Thus, the embodiments shall be regarded as illustrative rather than restrictive, and it should be understood that variations may be made in those embodiments by workers skilled in the art without departing from the scope of the present invention as set forth in the claims that follow, and equivalents thereof. In addition, the features of the different claims set forth below may be combined in various ways in further accordance with the present invention.

What is claimed is:

1. A method of accurately and non-invasively measuring a flow of mixture of liquid and particles comprising the steps of:
transmitting an ultrasound beam through the mixture and receiving any reflected ultrasound beams;
detecting with a detector any reflected ultrasound beams; and
analyzing the spectrum of the reflected ultrasonic beams including the steps of:
calculating a spectral intensity by performing a discrete Fourier transform (FT) of an output of the detector and processing the results of the Fourier transform to obtain the spectral intensity which is the square of the spectral amplitude of the FT results; and
multiplying the spectral intensity by the corresponding frequency of the spectral intensity of the output; and
summing the resulting products for all frequencies of the Fourier transform to form the numerator of a quotient; and
separately summing the spectral intensity for all frequencies of the Fourier transform to form the denominator of a quotient; and
obtaining a quotient from the numerator and denominator which is proportional to a total flow of the particles.

2. The method of claim 1, wherein the mixture comprises blood.

3. A method of accurately and non-invasively measuring a flow of mixture of liquid and particles comprising the steps of:
transmitting an ultrasound beam through the mixture and receiving any reflected ultrasound beams;
detecting with a detector any reflected ultrasound beams and outputting a detector output signal; and
analyzing the spectrum of the reflected ultrasonic beams including the step of:
determining a maximum frequency of the detector output signal which is representative of a maximum particle velocity; and
calculating a total flow of the particles based on the maximum particle frequency.

4. The method of claim 3, wherein the mixture comprises blood.

5. The method of claim 4, wherein the maximum frequency of the detector output signal is determined by:
performing a Fourier transform of the detector output signal to yield an FT output; and
calculating a spectral edge by detecting the frequency where a spectral amplitude of the FT output substantially decreases over a short frequency interval, the spectral edge representing the maximum particle velocity.

6. The method of claim 5, further comprising the step of:
performing frequency to flow rate scaling to determine the total flow of the particles.

7. The method of claim 4, wherein the mixture comprises blood.

8. A method of accurately and non-invasively measuring a flow of mixture of liquid and particles comprising the steps of:
providing an apparatus that transmits an ultrasound beam and receives any reflected ultrasound beam;
transmitting the ultrasound beam using the apparatus;
detecting with a detector any reflected ultrasound beam; and
analyzing the spectrum of the reflected ultrasonic beam including the steps of:
calculating a spectral intensity by performing a discrete Fourier transform of an output of the detector by performing the steps of;
processing the results of the Fourier transform to obtain the spectral intensity;
multiplying the spectral intensity by a frequency-proportionate weighting factor and summing the spectral intensity to produce a numerator of a quotient; and
summing the spectral intensity with a unity weighting factor to produce a denominator of the quotient, wherein the quotient is representative of a total flow of the particles.
